(19) United States
(12) Patent Application Publication
(10) Pub. No.: US 2004/0234497 A1
(43) Pub. Date: Nov. 25, 2004

(54) Hyaluronic acid containing bioconjugates:targeted delivery of anti-cancer drugs to cancer cells

(76) Inventors: Yi Luo, Salt Lake City, UT (US); Glenn D. Prestwich, Salt Lake City, UT (US); Jindrich Kopecek, Salt Lake City, UT (US); Zheng-Rong Lu, Salt Lake City, UT (US)

Correspondence Address: NEEDLE & ROSENBERG, P.C., SUITE 1000, 999 PEACHTREE STREET, ATLANTA, GA, 30309-3915, US

(21) Appl. No.: 10/476,824

(22) PCT Filed: May 6, 2002
(86) PCT No.: PCT/US02/14402

Related U.S. Application Data
(60) Provisional application No. 60/289,038, filed on May 4, 2001.

(51) Int. Cl. A61K 38/19; A61K 31/728

(52) U.S. Cl. 424/85.1; 514/34; 514/183; 514/54; 514/283; 514/449; 530/351; 536/53

(57) ABSTRACT

A cell-targeted polymeric drug delivery system was designed based on the specific interaction between hyaluronic acid (HA) and its cell surface receptors overexpressed on cancer cell surface. The invention relates to compounds composed of a carrier molecule, 5 wherein the carrier molecule contains at least one residue of an anti-cancer agent and at least one residue of a hyaluronic acid. The invention also relates to methods of making and using the compounds thereof.

### CROSS-REFERENCE TO RELATED APPLICATIONS

This application claims priority upon U.S. application Ser. No. 60/289,038, filed on May 4, 2001, which is herein incorporated by reference in its entirety.

### I. ACKNOWLEDGEMENTS

This invention was made with government support under Grants DAMD 17-9A-1-8254 provided by the Department of Army. The government has certain rights in the invention.

### II. BACKGROUND OF THE INVENTION

A major challenge in cancer therapy is to selectively deliver small molecule anti-cancer agents to tumor cells. One of the most promising methods involves the combination or covalent attachment of the cytotoxin with a macromolecular carrier¹. Many kinds of drug carriers, including soluble synthetic and natural polymers², liposomes³, microspheres⁴, and nanospheres^{5,6} have been employed to increase drug concentration in target cells. By altering the pharmacokinetic distribution of drugs, a sustained therapeutic concentration can be maintained at tolerable doses. Water-soluble polymer-anti-cancer drug conjugates seem to offer great potential because they can traverse compartmental barriers in the body⁷ and therefore gain access to a greater number of cell-types. A variety of water-soluble polymers, such as human serum albumin (HSA)², dextran⁸, lectins⁹, poly(ethylene glycol) (PEG)¹⁰, poly(styrene-co-maleic anhydride) (SMA)¹¹, poly(N-hydroxylpropylmethacrylamide) (HPMA)¹², and poly(divinylether-co-maleic anhydride) (DIVEMA)¹³ have been used to prepare polymeric anti-cancer prodrugs for cancer treatment. Such drug-polymer conjugates have demonstrated good solubility in water, increased half-life in the body, and high anti-tumor effects. For example, poly (styrene-co-maleic acid)-neocarzinostain conjugate (SMANCS) was approved for the treatment of liver cancer in Japan^{11,14}. The linking of doxorubicin to HPMA (HPMA-DOX) gives a new prodrug with improved in vitro tumor retention, a higher therapeutic ratio, and avoidance of multi-drug resistance¹². This system has passed the Phase I clinical trial and is currently in Phase II trials against ovarian cancer The conjugate of HPMA copolymer-camptothecin was also pre-clinically evaluated and is now in Phase I^{16,17}.

Anti-cancer polymer-drug conjugates can be divided into two targeting modalities: passive and active. The biological activity of the passive targeting drug delivery systems is based on the anatomical characteristics of tumor tissue, and allows polymeric prodrugs to more easily permeate tumor tissues and accumulate over time. This is one of the chief reasons for the success of polymeric drugs, it is often referred to as the enhanced permeability and retention (EPR) effect. Maeda proved that macromolecules can accumulate more efficiently in solid tumors than free drugs¹¹. Active targeting drug delivery systems can be achieved using specific interactions between receptors on the cell surface and the introduction of targeting moieties conjugated to the polymer backbone. In this way, active therapeutic agents conjugated to polymers can be selectively transported to tumor tissues. The active approach therefore takes advantage of the EPR effect, but further increases therapeutic index through receptor-mediated uptake by target cancer cells. Previous studies showed that N-acylated galactosamine¹⁸ and monoclonal antibodiy fragments¹⁹ were valuable targeting moieties for HPMA-DOX conjugates, selectively increasing the cytotoxicity of the polymer-drug conjugates to tumor cells.

Hyaluronic acid (HA, also known as hyaluronan, FIG. 1), a linear polysaccharide of alternating D-glucuronic acid (GlcUA) and N-acetyl-D-glucosamine (GlcNAc) units, is present in the extracellular matrix, the synovial fluid of joints, and the scaffolding that comprises cartilage²⁰ It is an immunoneutral building block for preparing biocompatible and biodegradable biomaterials^{21,25}, and has been employed as both a vehicle and an angiostatic agent in cancer therapy²⁶⁻²⁸. Mitomycin C and epirubicin were coupled to HA by carbodiimide chemistry and the HA-mitomycin adduct was selectively toxic to a lung carcinoma xenograft²⁹. Recently, the use of mild hydrazide chemistry to prepare an HA-Taxol® bioconjugate^{30,31} has been described, which showed good selectivity in cell culture studies. It is evident that directly correlates uptake with cytotoxicity using a fluorescently-labeled HA-Taxol® derivative, and it was demonstrated that toxicity is due to hydrolytic release of the parent drug.

HA serves a variety of functions within the extracellular matrix, including direct receptor-mediated effects on cell behavior. These effects occur via intracellular signaling pathways in which HA binds to, and is internalized by, cell surface receptors. Several cell membrane-localized receptors (HA binding proteins) have been identified including: CD44, RHAMM, IVd4, and the liver endothelial cell clearance receptor^{32,35}. HA-protein interactions play crucial roles in cell adhesion, growth and migration^{36,38}, and HA acts as a signaling molecule in cell motility, inflammation, wound healing, and cancer metastasis³⁹. The structure and regulation of HA receptors⁴⁰ is a growing area of structural and cellular biology that is critical to understanding how HA-protein interactions enhance metastasis.

Most malignant solid tumors contain elevated levels of HA⁴¹, and these high levels of HA production provide a matrix that facilitates invasion⁴². Clinically, high HA levels correlate with poor differentiation and decreased survival rate in some human carcinomas. HA is an important signal for activating kinase pathways^{43,44} and regulating angiogenesis in tumors⁴⁵. HA internalization is mediated via matrix receptors, including CD44, which is a transmembrane receptor that can communicate cell-matrix interactions into cells and can alter the matrix in response to intracellular signals. The pathological enrichment of HA in tumor tissues suggests that manipulation of the interactions between HA and its receptors could lead to dramatic inhibition of growth or metastasis of several types of tumor. Antibodies to CD44, soluble forms of CD44 or RHAMM, HAse, and oligomers of HA have all been used effectively to inhibit tumor growth or metastasis in animal models.

In addition to elevated HA in the environment surrounding tumors, most malignant cell-types overexpress CD44 and RHAMM. As a result, malignant cells with the highest metastatic potential often show enhanced binding and internalization of HA⁴⁶. Apparently, such cells can effectively breach the tumor-associated HA barrier by binding, internalizing, and degrading this glycosaminoglycan. Cell culture experiments suggest that CD44-HA interactions occur in vivo and are likely to be responsible for retention of HA-enriched matrices. Thus, HA can bind to the cell surface via interactions with CD44, and a portion subsequently undergoes endocytosis. In addition, internalization of ³H-labeled HA revealed that intracellular degradation of HA occurs within a low pH environment, such as that of lysosome. Targeting of anti-cancer agents to tumor cells and tumor metastases can be accomplished by receptor-mediated uptake of bioconjugates of anti-cancer agents conjugated to HA²⁹⁻³¹, followed by the release of free drugs through the degradation of HA in cell compartments. Isoforms of HA receptors, CD44 and RHAMM are over-expressed in transformed human breast epithelial cells⁴⁷, human ovarian tumor cells⁴⁸, and other cancers^{49,50}.

### III. SUMMARY OF THE INVENTION

In accordance with the purposes of this invention, as embodied and broadly described herein, this invention, in one aspect, relates to compounds comprising an anti-cancer agent, a carrier molecule, and hyaluronic acid or a derivative thereof, wherein the anti-cancer agent, the carrier molecule, and the hyaluronic acid or a derivative thereof are attached to one another via a covalent bond. The invention also relates to methods of making and using these compounds.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### IV. BRIEF DESCRIPTION OF THE DRAWINGS

The accompanying drawings, which are incorporated in and constitute a part of this specification together with the description serve to explain the principles of the invention.

FIG. 1 shows a tetrasaccharide fragment of HA with the repeating disaccharide units.

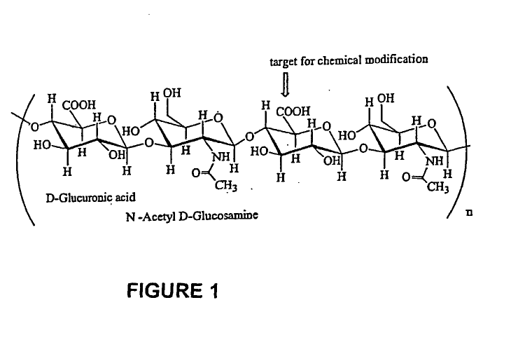

FIG. 2 shows the possible attachments of the anti-cancer agent, the carrier molecule, and the hyaluronic acid or derivative thereof to one another.

FIG. 3 shows a synthesis of HA-DOX conjugates.

FIG. 4 shows a structure of HPMA-HA-DOX conjugates.

FIG. 5 shows data for an In vitro cytotoxicity of HPMA-HA-DOX conjugates against HBL-100 human breast cancer cells. Cell viability of HBL-100 cells as function of DOX equivalent concentration. The cytotoxicity of polymer conjugates (targeted and non-targeted) were determined using MTT assay.

FIG. 6 shows a binding of targeted HPMA-HA-DOX conjugate on human ovarian cancer SK-OV-3 cells surface, (a) transmission image; (b) fluorescence (50 μg/ml HA equivalent of HPMA-HA-DOX at 0° C. for 2hr).

FIG. 7 shows a time course of internalization of targeted HPMA-HA-DOX conjugates (50 μg/ml HA equivalent) on human ovarian cancer SK-OV-3 cells in comparison with non-targeted HPMA-DOX conjugate.

FIG. 8 shows in vitro cytotoxicity of DOX, non-targeted HPMA-DOX conjugate, targeted HPMA-HA-DOX with 17% and 36% HA loading against human prostate cancer cell-line DU-145.

### V. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific compositions, or to particular formulations, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that if a particular value is disclosed, then “about” that value is also disclosed even if it is not specifically recited. For example, if the value 10 is disclosed, then “about 10” is also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Reference will now be made in detail to the present preferred embodiments of the invention, an examples of which are illustrated in the accompanying drawings. Wherever possible, the same reference numbers are used throughout the drawings to refer to the same or like parts.

A. Compounds

Free anti-cancer agents typically enter cells via passive, or non-energy-requiring, mechanisms. This can lead to loss of drug efficacy as a result of the action of the evolution of the multidrug resistance gene (MDR) due to the P-glycoprotein product, which pumps free drugs out of the cell. Polymeric drugs enter cells by pinocytosis or endocytosis rather than membrane fusion, and polymeric drugs are less susceptible to inducing MDR. Polymeric drugs also exhibit enhanced permeability and retention (EPR), e.g., the leaky vasculature of tumors allows macromolecular drugs to “concentrate” in the tumor tissues. The EPR effect improves targeting to malignant cells over normal cells; however, the macromolecular drugs have reduced overall cytotoxicity to all cells relative to the free drug. Thus, polymeric (macromolecular) drugs have reduced systemic side effects relative to the free drug. Furthermore, the cytotoxicity to cancer cells can be enhanced, without increasing toxicity to normal cells, by using a targeting agent, e.g., an antibody to a tumor antigen. The compounds of the present invention possess these attributes, increasing the delivery of anticancer agents. In addition the disclosed compositions enhances both the targeting to a specific cell as well as the uptake by the targeted cancer cells relative to other targeting strategies for small molecule or macromolecular anticancer drugs.

Disclosed are compounds that can be used, for example, in anti-cancer therapies. These compounds typically increase or alter the targeted delivery of anticancer compounds or other therapeutic compounds. Typically these compounds will comprise an anti-cancer agent, some other type of carrier molecule, and a molecule, such as HA.

Disclosed are compounds comprising an anti-cancer agent, a carrier molecule, and hyaluronic acid or a derivative thereof, wherein the anti-cancer agent, the carrier molecule, and the hyaluronic acid or a derivative thereof are attached to one another via a covalent bond.

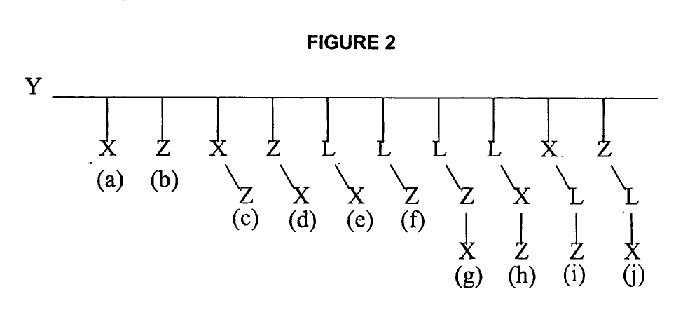

There are a number of different ways the anti-cancer agent, the carrier molecule, and the hyaluronic acid or derivative thereof can be attached to one another by a covalent bond. A non-limiting set of exemplary linkages are depicted in FIG. 2. In FIG. 2, X is the tethered moiety of the anti-cancer agent, Y is the tethered moiety of the carrier molecule, and Z is the tethered moiety of hyaluronic acid or the derivative thereof. A “tethered moiety ” can be any portion of a starting molecule that becomes a portion of a molecule produced in a reaction with the starting molecule. For example, hyaluronic acid could be depicted as Z-COOH. If Z-COOH was reacted with another molecule, such as A, and the product formed from this reaction was Z-A, then Z would be considered a tethered moiety . Likewise, if a subpart of Z was considered Z′ and the reaction of Z-COOH and A produced Z′-A, then Z′ would also be considered a tethered moiety . When Z-COOH reacts with a dihydrazide to produce a derivative of hyaluronic acid, Z remains the same and is part of the derivatized hyaluronic acid. In other words, Z is the tethered moiety of the original hyaluronic acid. In one embodiment, the anti-cancer agent, the carrier molecule, and the hyaluronic acid or derivative thereof can be directly attached to one another. For example, the anti-cancer agent and/or hyaluronic acid or derivative thereof are directly attached to the carrier molecule via a covalent bond (FIGS. 2(a) and (b), respectively). In another embodiment, the anti-cancer agent is directly attached to the carrier molecule via a covalent bond, and hyaluronic acid or derivative thereof is directly attached to the anti-cancer agent residue. Alternatively, hyaluronic acid or a derivative thereof is directly attached to the carrier molecule via a covalent bond, and the anti-cancer agent is directly attached to the hyaluronic acid or derivative thereof. These embodiments are depicted in FIGS. 2(c) and (d), respectively.

In another embodiment, the anti-cancer agent, carrier molecule, and the hyaluronic acid or a derivative thereof can be indirectly attached to one another by a linker. These embodiments are depicted in FIGS. 2(e)-(j). For example, in FIG. 2(e), the anti-cancer agent is indirectly attached to the carrier molecule by a linker (L denotes the residue of the linker), wherein the anti-cancer agent and the carrier molecule are individually and directly attached to the linker via a covalent bond. Examples of linkers include, but are not limited to, succinates, disulfide-containing compounds, and diol-containing compounds. The linkers may also include short peptides with specific targeting sequences for lysosomes and for lysosomal degradation, such as Gly-Phe-Leu-Gly. Other examples include, for prostate cancer, linkages targeted to prostate cells and to a prostate-specific antigen (PSA), which has sequence-specific proteolytic capabilities. In this example, PSA hydrolyzes His-Ser-Ser-Lys-Leu-Gln and glutaryl4-hydroxyprolyl-Ala-Ser-cyclohexaglycyl-Gln-Ser-Leu.

The linkers are typically cleavable so that the anti-cancer agent can be released, for example, under reducing conditions, oxidizing conditions, or by hydrolysis of an ester, amide, hydrazide, or similar linkage forms the covalent bond between the linker and the anti-cancer agent. Additionally, the type of linker may augment the selective cytotoxicity (and thus improve the therapeutic index) aspect by permitting selective release of the anti-cancer agent inside the cells targeted by the targeting moiety (carrier molecule or HA).

The invention also contemplates further attaching an anti-cancer agent to hyaluronic acid or a derivative thereof that is indirectly attached to the carrier molecule via a linker. Additionally, it is possible to attach hyaluronic acid or a derivative thereof to an anti-cancer agent that is indirectly attached to the carrier molecule via a linker. These embodiments are depicted in FIGS. 2(g) and (h), respectively.

In another embodiment, the anti-cancer agent and hyaluronic acid or a derivative thereof can be attached to one another via a linker molecule. These embodiments are depicted in FIGS. 2(i) and (j). In FIGS. 2(i) and (j), the anti-cancer agent and the hyaluronic acid or derivative thereof, respectively, are directly attached to the carrier molecule.

The anti-cancer agent, the carrier molecule, and the hyaluronic acid or derivatives thereof used to produce the compounds are discussed below.

1. Anti-cancer Agent

Any anti-cancer agent can be directly or indirectly attached to the carrier molecule and the hyaluronic acid or derivatives to be aided in transport across the cellular membranes. There are many anti-cancer agents known in the art. In one embodiment, the anti-cancer agent is any small molecule that targets intracellular function, such as protein kinase inhibitors including but not limited to Gleevac. In another embodiment, radionuclides including, but not limited to, I-131, Y-90. In-111, Tc-99m can beused. In another embodiment, Gd+3 compounds can be used. In yet another embodiment, meso e-chlorin and cis-platin derivatives can be used as the anti-cancer agent. A partial list of anti-cancer agents that can be used with the disclosed compositions can be found in, for example, U.S. Pat. No. 5,037,883, which is herein incorporated by reference as well as any publications and patents, or patent applications, cited therein which contain anti-cancer agents. Other anticancer agents, such as, cytotoxic agent, a chemotherapeutic agent, a cytokine, antitubulin agents, and a radioactive isotope, can also be used in the disclosed compounds. Anticancer agents, such as, vincristine, vinblastine, vinorelbine, and vindesine, calicheamicin, QFA, BCNU, streptozoicin, and 5-fluorouracil, neomycin, podophyllotoxin(s), TNF-alpha, .alphaᵥbeta₃ colchicine, taxol, a combretastatin antagonists, calcium ionophores, calcium-flux inducing agents, and any derivative or prodrug thereof can also be used herein. U.S. Pat. Nos. 6,348,209, 6,346,349, and 6,342,221 are also disclosed for agents related to anti-cancer compounds. In certain embodiments, the anti-cancer agent comprises 5-fluorouracil, 9-aminocamptothecin, or amine-modified geldanomycin. In another embodiment, the anti-cancer agent is doxorubicin. In yet another embodiment the anticancer agent can be Taxol®. However, anti cancer agents, such as the anti-growth factor receptor antibodies (e.g., Herceptin), are understood to not typically have a need for transport across a cell membrane, and therefore, would typically be used in combination with the disclosed compounds and compositions.

2. Carrier Molecule

Any carrier molecule can be used. Typcially carrier molecules will be polymer molecules. Typically the carrier molecule is a large macromolecule of at least 5,000 daltons. The carrier molecule can range from 2,000 daltons to 25,000 daltons, or from 25,000 daltons to 100,000 daltons, or from 100,000 daltons to 1,000,000 daltons. It is preferred that the carrier molecule be in the range of 10,000 to 25,000 daltons. The carrier molecule typically aids in the transport of anti-cancer agent across the cell membrane. Thus, when the anti-cancer agent is directly or indirectly attached to the carrier molecule it typically crosses a cell membrane better than the anti-cancer agent alone. There are numerous carriers and macromolecular carriers known in the art that will function as the carrier molecule. Examples of carrier molecules are also described in, for example, U.S. Pat. Nos: 5,415,864 for “Colonic-targeted oral drug-dosage forms based on crosslinked hydrogels containing azobonds and exhibiting PH-dependent swelling;” 5,258,453 for “Drug delivery system for the simultaneous delivery of drugs activatable by enzymes and light;” 5,037,883 for “Synthetic polymeric drugs;” 4,074,039 for “Hydrophilic N,N-diethyl acrylamide copolymers;” 4,062,831 for “Copolymers based on N-substituted acrylamides, N-substituted methacrylamides and N,N-disubstituted acrylamides and the method of their manufacturing;” 3,997,660 for “Soluble hydrophilic polymers and process for producing the same;” 3,931,123 for “Hydrophilic nitrite copolymers;” and 3,931,111 for “Soluble hydrophilic polymers and process for processing the same” each of which is individually and specifically herein incorporated by reference at least for material related to carriers. It is understood that in certain embodiments, the carrier does not include HA or derivatives thereof

In one embodiment, the carrier molecule comprises a polymer produced by the polymerization of an ethylenically unsaturated monomer. Examples of monomers include, but are not limited to, acrylates and methacrylates. In one embodiment, the carrier molecule is a polymer produced from the polymerization of N-(2-hydroxypropoyl)methacrylamide, which is referred to herein as HPMA.

3. Hyaluronic Acid and Derivatives Thereof

The third component is hyaluronic acid (HA), a macromolecule having the properties of hyaluronic acid, and derivatives of hyaluronic acid. There are many uses for and derivatives of HA, some of which are disclosed in U.S. Pat. Nos. 5,616,568 and 5,652,347 and U.S. Provisional Application Nos. 60/116,021 and 60/218,725, which are herein incorporated by reference as well as the publications, patents, and patent applications cited therein at least for material related to hyaluronic acids. In one embodiment, the hyaluronic acid is modified with a dihydrazide compound such as adipic dihydrazide.

Hyaluronic acid is a polysaccharide of at least 4 disaccharide repeat units of HA, e.g., at least 1,000 daltons. HA and derivatives thereof can range from 1,000 daltons to 10,000 daltons, or from 10,000 daltons to 100,000 daltons, or from 100,000 daltons to 1,000,000 daltons. It is preferred that HA and its derivatives be at least 1,000 daltons. In one embodiment, the lower limit of the molecular weight is, 1000, 2000, 3000, 4000, 5000, 6000, 7000, 8000, 9000, or 10,000, and the upper limit is 100,000, 200,000, 300,000, 400,000, 500,000, 600,000, 700,000, 800,000, 900,000, or 1,000,000, where any lower limit can be combined with any upper limit. Hyaluronic acid typically aids in the transport of the anti-cancer agent across the cell membranes through an active mode of transport.

4. Efficiency and Specificity of Uptake by the Cells

The disclosed compounds can be characterized in that they allow for the uptake of anti-cancer agents by cells using typically different mechanisms than used by the anti-cancer agent alone. This efficiency can be measured in a number of ways. There are many ways to determine whether the efficiency and/or specificity of the uptake is increased by hyaluronic acid and/or the carrier molecule. For example, one can block the HA mediated transport and look at the change in saturation of the cells. One can do this by performing the assays with saturating HA present, using HA specific antibodies which block the HA function, using cells without HA receptors, and using cells that over express HA receptors like cancer cells. Typical increases of efficiency and/or specificity can be greater than or equal to at least 2 fold, 5 fold, 10 fold, 25 fold, 50 fold, 100 fold, 500 fold, 1000, fold 5000 fold or 10,000 fold.

The compounds have greater specificity for uptake and retention in the targeted cancer cells. This increased specificity is consistent with the specific hyaluronic acid receptors which import hyaluronic acid into cells. Typically disclosed compounds have a 5 to 100 fold greater specificity than either the anti-cancer-carrier molecule or anti-cancer-hyaluronic acid systems. This specificity can be assayed in a number of ways. For example, the intrinsic fluorescence of the anti-cancer agent doxorubicin may be observed directly by fluorescence microscopy in anti-cancer agent-carrier molecule systems and the disclosed compounds. The presence of hyaluronic acid in the disclosed compounds results in increases of 5 to 50 fold of the anti-cancer agent present inside prostate, ovarian, colon, or breast cells as well as other cells, for example, (among others). melanoma, bladder, lung, and gastrointestinal tumors. have also been described.

B. Method of Making Compounds

The compounds of the invention can be prepared using techniques known in the art. As described, there are three components used to produce the compounds: the anti-cancer agent, the carrier molecule, and hyaluronic acid or a derivative thereof. Any of the components previously described can be reacted with one another in any possible combination to produce the compounds of the invention. The invention also contemplates the use of two or more anti-cancer agents, carrier molecules, or hyaluronic acid or its derivatives thereof when producing the compounds of the invention. In addition, it is sometimes preferred to couple (i.e., react) two of the three components together to produce a new reaction product or intermediate, then chemically connect the intermediate with the third component. For example, the anti-cancer agent can react with the carrier molecule to produce an anti-cancer/carrier molecule. Similarly, the anti-cancer agent can react with hyaluronic acid or a derivative thereof to produce an anti-cancer/hyaluronic acid molecule, and hyaluronic acid or a derivative thereof can react with the carrier molecule to produce a hyaluronic acid/carrier molecule. Each of these intermediates can be reacted with an individual component (e.g., the reaction of anti-cancer/hyaluronic acid molecule with carrier molecule) or, alternatively, each of the intermediates can react with one another to produce the compound (e.g., reaction of anti-cancer/hyaluronic acid molecule with the anti-cancer/carrier molecule). In one embodiment, the compound can be produced by (1) reacting the anti-cancer agent with the carrier molecule to produce a carrier/anti-cancer molecule and (2) reacting the carrier/anti-cancer molecule with hyaluronic acid or the derivative thereof. For example, the carrier molecule HPMA is reacted with doxorubicin (DOX) to produce HPMA-DOX, then HPMA-DOX is reacted with hyaluronic acid modified with adipic dihydrazide to produce HPMA-DOX-HA. It should be noted that the reaction requires compatible reactive functionalities and generally includes a linker connecting the two tetherable moieties.

In another embodiment, the compound can be produced by (1) reacting the anti-cancer agent with hyaluronic acid or the derivative thereof to produce an anti-cancer/hyaluronic acid molecule; (2) reacting the anti-cancer agent with the carrier molecule to produce a carrier/anti-cancer molecule; and (3) reacting the anti-cancer/hyaluronic acid molecule with the carrier molecule/anti-cancer molecule. For example, hyaluronic acid is reacted with doxorubicin to produce HA-DOX, then HA-DOX is subsequently reacted with HPMA-DOX to produce HA-DOX-HPMA.

As described above, the anti-cancer agent, carrier molecule, and hyaluronic acid can be attached to one another directly or indirectly via a linker. In addition, the attachment of each component to one another can vary depending upon the types of components selected and the order in which the components are permitted to react with one another.

The invention also contemplates that two or more compounds can be produced simultaneously when the anti-cancer agent, the carrier molecule, and the hyaluronic acid or a derivative thereof are reacted with one another. Thus, it is possible to produce compositions or mixtures of compounds depending upon the type and amount of starting materials that are used. In one embodiment, the molecular weight of the carrier molecule and/or the hyaluronic acid or its derivatives will vary for each compound in the composition. In another embodiment, the attachment of the anti-cancer agent, carrier molecule, and hyaluronic acid or its derivatives to one another may vary from one compound to another in the composition. In another embodiment, the anti-cancer agent may be modified once it is attached to the carrier molecule or hyaluronic acid or its derivative thereof. The invention also contemplates the formation of compositions composed of one or more compounds of the invention and free anti-cancer agent. For example, an excess of anti-cancer agent is used relative to the carrier molecule and/or the hyaluronic acid to produce these compositions.

C. Method of Using Compounds

The disclosed compounds can be used for targeted delivery of anti-cancer agents to cells. These compounds can be used thus, to treat a variety of disorders that require the delivery of anti-cancer or similar agents. It is understood that any of the compounds disclosed can be used in this way. Those of skill in the art understand the compounds will be administered in pharmaceutically acceptable forms and in doses wherein delivery occurs. Typically the compounds would be administered to patients in need of delivery of the anti-cancer agent or a similar compound. It is understood that the goal is delivery of the compound and that through delivery affect the cells of the patient in need of the anti-cancer agent or similar agent.

Disclosed herein the conjugated anti-cancer agents can be given to a subject. Any subject in need of receiving an anti-cancer agent can be given the disclosed conjugated anti-cancer agents. The subject can, for example, be a mammal, such as a mouse, rat, rabbit hamster, dog, cat, pig, cow, sheep, goat, horse, or primate, such as monkey, gorilla, orangutan, chimpanzee, or human.

Disclosed herein the conjugated anti-cancer agents can used for inhibiting cancer cell proliferation. Inhibiting cancer cell proliferation means reducing or preventing cancer cell growth. Inhibitors can be determined by using a cancer cell assay. For example, either a cancer cell line can be cultured on 96-well plates in the presence or absence of the conjugated anti-cancer agent or anti-cancer agent alone or anti-cancer agent prepared differently then the disclosed compositions (for example, just anticancer agent and carrier) for any set period of time. The cells can then be assayed. In certain embodiments the conjugated anti-cancer compounds are those that will inhibit 10% or 15% or 20% or 25% or 30% or 35% or 40% or 45% or 50% or 55% or 60% or 65% or 70% or 75% or 80% or 85% or 90% or 95% of growth relative to any of the controls as determined by the assay.

Disclosed are compositions which inhibit metastatic tumor formation in this type of assay disclosed herein, as well as compositions that reduce metastatic tumor formation by at least 10% or 15% or 20% or 25% or 30% or 35% or 40% or 45% or 50% or 55% or 60% or 65% or 70% or 75% or 80% or 85% or 90% or 95% of a control compound.

Disclosed herein the disclosed conjugated anti-cancer agents can be administered to cells and/or cancer cells which have HA receptors.

The disclosed compounds can be administered after performing a toxicity-abatement, or blocking step with, for example, chondroitin sulfate to increase the specificity of cancer cell uptake. See co-pending United States Provisional Application entitled “Preblocking with non-HA GAGs Increases Effectiveness of HA Conjugated Anticancer Agents” by Prestwich et al. filed on the same day as this application, which is herein incorporated by reference in its entirety for material relating to at least to chondroitin sulfate administration. The disclosed compositions can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Compounds disclosed herein may also be used for the treatment of precancer conditions such as cervical and anal dysplasias, other dysplasias, severe dysplasias, hyperplasias, atypical hyperplasias, and neoplasias.

1. Dosages

The dosage ranges for the administration of the compounds are those large enough to produce the desired effect in which delivery occurs. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary from about 1 mg/kg to 30 mg/kg in one or more dose administrations daily, for one or several days.

The dose, schedule of doses and route of administration may be varied, whether oral, nasal, vaginal, rectal, extraocular, intramuscular, intracutaneous, subcutaneous, or intravenous, to avoid adverse reaction yet still achieve delivery.

2. Pharmaceutically Acceptable Carriers

Any of the compounds can be used therapeutically in combination with a pharmaceutically acceptable carrier.

Pharmaceutical carriers are known to those skilled in the art. These most typically would be standard carriers for administration of compositions to humans, including solutions such as sterile water, saline, and buffered solutions at physiological pH. The compositions could also be administered intramuscularly or subcutaneously. Other compounds will be administered according to standard procedures used by those skilled in the art.

Molecules intended for pharmaceutical delivery may be formulated in a pharmaceutical composition. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed compositions can be administered intravenously, intraperitoneally, intramuscularly, subcutaneously, intracavity, or transdermally.

Preparations for parenteral administration include sterile aqueous or non-aqueous solutions, suspensions, and emulsions which may also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration may include powders or granules, suspensions or solutions in water or non-aqueous media, capsules, sachets, or tablets. Thickeners, flavorings, diluents, emulsifiers, dispersing aids or binders may be desirable.

The compositions as described herein can also be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

C. References

The following references may be referred to in the specification and each one is specifically herein incorporated by reference.

(1) Kim, K. H., Hirano, T., and Ohashi, S. Anti-cancer Polymeric Prodrugs, targetable. In Polymeric Materials Encyclopedia; J. C. Salamone, Ed.; CRC Press: Boca Raton, Fla., 1996; pp 272-285.

(2) Trouet, A., Masquelier, M., Baurain, R., and Campeneere, D. D. (1982) A covalent linkage between daunorubicin and proteins that is stable in serum and reversible by lysosomal hydrolases, as required for a lysosomotropic drug-carrier conjugate: in vitro and in vivo studies. Proc. Natl. Acad. Sci. USA 79, 626-629.

(3) Kim, S. (1993) Liposomes as carriers of cancer chemotherapy. Drugs 46, 618-638.

(4) Arshady, R. (1990) Albumin microspheres and microcapsules: methodology of manufacturing techniques. J. Controlled Release 14, 111-131.

(5) Kreuter, J. (1991) Nanoparticle-based drug delivery systems. J. Controlled Release 16, 169-176.

(6) Waser, P. G., Kreuter, J., Berger, S., Munz, K., Kaiser, E., and Pfluger, B. (1987) Localization of colloidal particles (liposomes, hexylcyanoacrylate nanoparticles and albumin nanoparticles) by histology and autoradiography in mice. Int. J Pharm. 39, 213-227.

(7) Cartlidge, S. A., Duncan, R., Lloyd, J. B., Kopeckova, R. P., and Kopecek, J. (1987) Soluble, crosslinked N-(2-hydroxypropyl) methacrylamide copolymers as potential drug carrier 3. Targeting by incorporation of galactosamine residue. Effect of route of administration. J. Controlled Release 4, 265-278.

(8) Molteni, L. Dextrans as drug carriers. In Drugs Carriers in Biology and Medicine; G. Gregoriadis, Ed.; Academic: London, 1979; pp 107.

(9) Molteni, L. Lectins as drug carriers. In Drugs Carriers in Biology and Medicine; G. Gregoriadis, Ed.; Academic Press: London, 1979; pp 43.

(10) Greenwald, R. B., Gilbert, C. W., Pendri, A., Conover, C. D., Xia, J., and Martinez, A. (1996) Drug delivery systems: water soluble taxol 2′-poly(ethylene glycol) ester prodrugs—design and in vivo effectiveness. J. Med. Chem. 39, 424-431.

(11) Maeda, H., Seymour, L. W., and Miyamoto, Y. (1992) Conjugates of anti-cancer agents and polymers: advantages of macromolecular therapeutics in vivo. Bioconjugate Chem. 3, 351-362.

(12) Minko, T., Kopeckova, P., Pozharov, V., and Kopecek, J. (1998) HPMA copolymer bound adriamycin overcomes MDRI gene encoded resistance in a human ovarian carcinoma cell line. J. Controlled Release 54, 223-233.

(13) Hirano, T., Ohashi, S., Morimoto, S., and Tsuda, K. (1986) Synthesis of antitumor-active conjugates of adriamycin or daunomycin with the copolymer of divinyl ether and maleic anhydride. Makromol. Chem. 187, 2815-2824.

(14) Maeda, H., Ueda, M., Morinaga, T., and Matsumoto, T. (1985) Conjugation of poly(styrene-co-maleic acid) derivatives to the antitumor protein neocarzinostatin: pronounced improvements in pharmacological properties. J. Med. Chem. 28, 455-461.

(15) Vasey, P. A. e. a. (1999) Phase I clinical and pharmacokinetics study of PK1[N-(2-hydroxypropyl)methacrylamide copolymer doxorubicin]: first member of a new class of chemotherapeutic agents-drug-polymer conjugates. Clin. Cancer Res. 5, 83-94.

(16) Ciaolfa, V. R., Zamai, M., Fiorino, A., Frigerio, E., d'Argy, R., Ghiglieri, A., Farao, M., Angelucci, F., and Suarato, A. In Ninth international symposium on recent advances in drug delivery systems; Salt lake City, Utah, USA, 1999; pp 46.

(17) Ciaolfa, V. R., Zamai, M., Fiorino, A., Frigerio, E., Pellizzoni, C., d′Argy, R., Ghiglieri, A., Castelli, M. G., Farao, M., Pesenti, M., Gigli, F., Angelucci, F., and Suarato, A. (2000) Polymer-bound camptothecin: initial biodistribution and antitumor activity studies. J. Controlled Release 65, 105-119.

(18) Seymour, L. W., Ferry, D. R., Boivin, C., Julyan, P., Doran, J., David, M., Anderson, D., Christodoulou, C., Young, A. M., Kerr, D. J., and Hesslewood, S. In Eighth international symposium on recent advances in drug delivery systems; Salt lake City, Utah, USA, 1997; pp 132-135.

(19) Lu, Z.-R., Kopecekova, P., and Kopecek, J. (1999) Polymerizable Fab' antibody fragments for targeting of anti-cancer drugs. Nature Biotechnology 17, 1101-1104.

(20) Laurent, T. C., Laurent, U. B. G., and Fraser, J. R. E. (1995) Functions of hyaluronan. Ann. Rheum. Dis. 54, 429-432.

(21) Luo, Y., Kirker, K. R., and Prestwich, G. D. (2000) Cross-linked hyaluronic acid hydrogel films: new biomaterials for drug delivery. J. Controlled Release 69, 169-184.

(22) Vercruysse, K. P., and Prestwich, G. D. (1998) Hyaluronate derivatives in drug delivery. Crit. Rev. Ther. Drug Carr. Syst. 15, 513-555.

(23) Prestwich, G. D., Marecak, D. M., Marecek, J. F., Vercruysse, K. P., and Ziebell, M. R. Chemical modification of hyaluronic acid for drug delivery, biomaterials, and biochemical probes. In The Chemistry, Biology, and Medical Applications of Hyaluronan and its Derivatives; T. C. Laurent, Ed.; Portland Press: London, 1998; pp 43-65.

(24) Prestwich, G. D., Luo, Y., Ziebell, M. R., Vercruysse, K. P., Kirker, K. R., and MacMaster, J. S. Chemically-modified hyaluronan: New biomaterials and probes for cell biology. In New Frontiers in Medical Sciences: Redefining Hyaluronan; G. Abatangelo, Ed.; Portland Press: London, 2000; pp 181-194.

(25) Freed, L. E., Vunjak-Novakovic, G., Biron, R. J., Eagles, D. B., Lesnoy, D. C., Barlow, S. K., and Langer, R. (1994) Biodegradable polymer scaffolds for tissue engineering. Bio/Technology 12, 689-693.

(26) Alam, C. A. S., Seed, M. P., and Willoughby, D. A. (1995) Angiostasis and vascular regression in chronic granulomatous inflammation induced by diclofenac in combination with hyaluronan in mice. J. Pharm. Pharmacol. 47, 407-411.

(27) Falk, R. E. In PCT Int. Appl. WO9740841; 1997; pp.

(28) Ziegler, J. (1996) Hyaluronan seeps into cancer treatment trials. J. Nat. Cancer Inst. 88, 397-399.

(29) Akima, K., Ito, H., Iwata, Y., Matsuo, K., Watari, N., Yanagi, M., Hagi, H., Oshima, K., Yagita, A., Atomi, Y., and Tatekawa, I. (1996) Evaluation of antitumor activities of hyaluronate binding antitumor drugs: synthesis, characterization and antitumor activity. J. Drug Targeting 4, 1.

(30) Luo, Y., and Prestwich, G. D. (1999) Synthesis and selective cytotoxicity of a hyaluronic acid-antitumor bioconjugate. Bioconjugate Chem. 10, 755-763.

(31) Luo, Y., Ziebell, M. R., and Prestwich, G. D. (2000) A hyaluronic acid-taxol antitumor bioconjugate targeted to cancer cells. Biomacromolecules 1, 208-218.

(32) Turley, E. A., Belch, A. J., Poppema, S., and Pilarski, L. M. (1993) Expression and function of a receptor for hyaluronan-mediated motility on normal and malignant lymphocytes-B. Blood 81, 446-453.

(33) Toole, B. P. (1997)Hyaluronan in morphogenesis. J. Intern. Med. 242, 35-40.

(34) Rudzki, Z., and Jothy, S. (1997) CD44 and the adhesion of neoplastic cells. J. Clin. Pathol. Mol. Pathol. 50, 57-71.

(35) Lesley, J., Hyman, R., English, N., Catterall, J. B., and Turner, G. A. (1997) CD44 in inflammation and metastasis. Glycoconjugate J. 14, 611-622.

(36) Underhill, C. (1992) CD44—The Hyaluronan Receptor. J. Cell Sci. 103, 293-298.

(37) Turley, E. A. The role of a cell-associated hyaluronan binding protein in fibroblast behavior. In The Biology of Hyaluronan; C. Foundation, Ed.; J. Wiley & Sons, Ltd.: Chichester, UK, 1989; pp 121-137.

(38) Knudson, C. B., and Knudson, W. (1993) Hyaluronan-Binding proteins in development, tissue homeostasis, and disease. FASEB J 7, 1233-1241.

(39) Entwistle, J., Hall, C. L., and Turley, E. A. (1996) Hyaluronan receptors: regulators of signalling to the cytoskeleton. J. Cell Biochem. 61, 569-577.

(40) Day, A. J. (1999) The structure and regulation of hyaluronan-binding proteins. Biochem. Soc. Trans. 27, 115-121.

(41) Yeo, T. K., Nagy, J. A., Yeo, K. T., Dvorak, H. F., and Toole, B. P. (1996) Increased hyaluronan at sites of attachment to mesentery by CD44-positive mouse ovarian and breast tumor cells. Am. J. Pathol. 148, 1733-1740.

(42) Knudson, W. (1996) Tumor-associated hyaluronan: providing an extracellular matrix that facilitates invasion. Am. J. Pathol. 148, 1721-1726.

(43) Hall, C. L., Yang, B. H., Yang, X. W., Zhang, S. W., Turley, M., Samuel, S., Lange, L. A., Wang, C., Curpen, G. D., Savani, R. C., Greenberg, A. H., and Turley, E. A. (1995) Overexpression of the hyaluronan receptor RHAMM is transforming and is also required for H-ras transformation. Cell 82, 19-28.

(44) Nelson, R. M., Venot, A., Bevilacqua, M. P., Linhardt, R. J., and Stamenkovic, I. (1995) Carbohydrate-protein interactions in vascular biology. Annu. Rev. Cell Dev. Biol. 11, 601-631.

(45) Rooney, P., Kumar, S., Ponting, J., and Wang, M. (1995) The role of hyaluronan in tumour neovascularization (review). Int. J. Cancer 60, 632-636.

(46) Hua, Q., Knudson, C. B., and Knudson, W. (1993) Internalization of hyaluronan by chondrocytes occurs via Receptor-Mediated endocytosis. J. Cell Sci. 106, 365-375.

(47) Iida, N., and Bourguignon, L. Y.-W. (1997) Coexpression of CD44 variant (v10/ex14) and CD44S in human mammary epithelial cells promotes tumorigenesis. J. Cell. Physiol. 171, 152-160.

(48) Bourguignon, L. Y.-W., Zhu, H. B., Chu, A., Iida, N., Zhang, L., and Hung, M. C. (1997) interaction between the adhesion receptor, CD44, and the oncogene product, p185(HER2), promotes human ovarian tumor cell activation. J. Biol. Chem. 272, 27913-27918.

(49) Culty, M., Nguyen, H. A., and Underhill, C. B. (1992) The hyaluronan receptor (CD44) participates in the uptake and degradation of hyaluronan. J. Cell Biol. 116, 1055-1062.

(50) Culty, M., Shizari, M., Thompson, E. W., and Underhill, C. B. (1994) Binding and degradation of hyaluronan by human breast cancer cell lines expressing different forms of CD44: correlation with invasive potential. J. Cell. Physiol. 160, 275-286.

(51) Pouyani, T., and Prestwich, G. D. (1994) Functionalized derivatives of hyaluronic acid oligosaccharides—drug carriers and novel biomaterials. Bioconjugate Chem. 5, 339-347.

(52) Shih, L. B., Goldenberg, D. M., Xuan, H., Lu, H., Sharkey, R. M., and Hall, T. C. (1991) Anthracycline Immunoconjugates prepared by a site-specific linkage via an amino-dextran intermediate carrier. Cancer Res. 51, 4192-4198.

(53) Ogura, H., Nagai, S., and Takeda, K. (1980) A novel reagent (N-succinimidyl diphenylphosphate ) for synthesis of active ester and peptide. Tetrahedron Lett. 21, 1467-1468.

(54) Kopecek, J., Rejmanova, P., Strohalm, J., Ulbrich, K., Rihova, B., Chytry, V., Lloyd, J. B., and Duncan, R. In U.S. Patent; USA, 1991; pp.

(55) Omelyanenko, V. G., Kopecekova, P., Gentry, C., Shiah, J.-G., and Kopecek, J. (1996) HPMA copolymer-anti-cancer drug-OV-TL16 antibody conjugates. 1. Influence of the methods of synthesis on the binding affinity to OVCAR-3 ovarian carcinoma cells in vitro. J. Drug Target. 3, 357-373.

(56) Puttnam, D., and Kopecek, J. (1995) Polymer conjugates with anti-cancer activity. Adv. Polym. Sci. 122, 55-123.

(57) Rejmanova, P., Labsky, J., and Kopecek, J. (1977) Aminolyses of monomeric and polymeric p-nitrophenyl esters of methacryloylated amino acids. Makromol. Chem. 178, 2159-2168.

(58) Kokoshka, J. M., Ireland, C. M., and Barrows, L. R. (1996) Cell-based screen for identification of inhibitors of tubulin polymerization. J. Nat. Prod. 59, 1179-1182.

(59) Pouyani, T., and Prestwich, G. D. In U.S. Pat. No. 5,616,568; Research Foundation of SUNY: USA, 1997; pp.

(60) Kreil, G. (1995) Hyaluronidases—a group of neglected enzymes. Protein Sci. 4, 1666-1669.

(61) Chow, G., Knudson, C. B., Homandberg, G., and Knudson, W. (1995) Increased expression of CD44 in bovine articular chondrocytes by catabolic cellular mediators. J. Biol. Chem. 270, 27734-27741.

(62) Lesley, J., and Hyman, R. (1992) CD44 can be activated to function as an hyaluronic acid receptor in normal murine T-cells. Eur. J. Immunol. 22, 2719-2723.

(63) Lesley, J., English, N., Perschl, A., Gregoroff, J., and Hyman, R. (1995) Variant cell lines selected for alterations in the function of the hyaluronan receptor CD44 show differences in glycosylation. J. Exp. Med. 182, 431-437.

(64) Perschl, A., Lesley, J., English, N., Trowbridge, I., and Hyman, R. (1995) Role of CD44 cytoplasmic domain in hyaluronan binding. Eur. J. Immunol. 25, 495-501.

(65) Asplund, T., and Heldin, P. (1994) Hyaluronan receptors are expressed on human malignant mesothelioma cells but not on normal mesothelial cells. Cancer Res. 54, 4516-4523.

(66) Nakabayashi, H., Tsujii, H., Okamoto, Y., and Nakano, H. (1996) Fluorescence-labeled-hyaluronan loading test as an index of hepatic sinusoidal endothelial cell function in the rat. Int. Hepatol. Commun. 5, 345-353.

(67) Collis, L., Hall, C., Lange, L., Ziebell, M., Prestwich, G., and Turley, E. (1998) Rapid hyaluronan uptake is associated with enhanced motility: implications for an intracellular mode of action. FEBS Lett. 440, 444-449.

D. Examples

Disclosed herein the selective delivery of polymeric-antitumor agent conjugate to cancer cells can be markedly enhanced, and that overall doses could be reduced.

Cell-targeted hyaluronic acid (HA)-doxorubicin (DOX) biconjuagtes (HA-DOX), and N-(2-hydroxypropoyl)methacrylamide (HPMA) copolymer-DOX conjugates containing HA as a side chain (HPMA-HA-DOX) were synthesized based on the specific interaction between hyaluronic acid (HA) and its receptors overexpressed on cancer cell surface. Selective in vitro cell cytotoxicity was studied with three human cell-lines (HCT-116 colon tumor, HBL-100 breast cancer, and SK-OV-3 ovarian cancer). In addition, enhanced uptake of HPMA-HA-DOX conjugate was visualized by confocal fluorescence microscopy in comparison to non-targeted HPMA-HA-DOX system, providing compelling evidence for the uptake of the targeted conjugates through receptor-mediated pathway.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1 Doxyrubicin HA-HPMA

### a) Methods

(1) Reagents

Fermentation-derived HA (sodium salt, Mᵣ 1.5 MDa) was provided by Clear Solutions Biotechnology, Inc. (Stony Brook, N.Y.). 1-Ethyl-3-(3-(dimethylamino)-propyl)carbodiimide (EDCI), Adipic dihydrazide (ADH), succinic anhydride, anhydrous DMF, and triethylamine were purchased from Aldrich Chemical Co. (Milwaukee, Wis.). Testicular hyaluronidase (HAse), Dulbecco's phosphate-buffered saline (DPBS) and cell culture media were purchased from Sigma (St. Louis, Mo.). Doxorubicin (DOX) was a kind of gift from Dr. A. Suarato, Pharmacia-Upjoin, Milano, Italy. Fluorescence images were recorded on a Bio-Rad (Hercules, Calif.) MRC 1024 laser scanning confocal imaging system based on a Zeiss (Oberkochen, Germany) Axioplan microscope and a krypton/argon laser.

(2) Cell Lines.

HBL-100, a human breast cancer cell-line, was maintained in culture in high glucose D-MEM (Dulbecco's Modified Eagle Medium), which was supplemented with 10% γ-irradiated fetal bovine serum (FBS) and 1% sodium pyruvate; SK-OV-3, a human ovarian cancer cell-line was cultured in D-MEM/F12+10% FBS; HCT-116, a colon tumor cell-line, was maintained in culture in α-MEM (Minimal Essential Medium, Eagle)+10% FBS.

(3) Analytical Instrumentation.

All ¹H NMR spectral data were obtained using an NR-200 FT-NMR spectrometer at 200 MHz (IBM Instruments Inc.). UV-Vis spectra were recorded on a Hewlett Packard 8453 UV-Vis diode array spectrophotometer (Palo Alto, Calif.). HA was characterized by gel permeation chromatography (GPC) was on the following system: Waters 515 HPLC pump,

Waters 410 differential refractometer, and Waters™ 486 tunable absorbance detector. Waters Ultrahydrogel 250 and 2000 columns (7.8 mm ID×30 cm) (Milford, Mass.) were used for GPC analysis, the eluent was 150 mM pH 6.5 phosphate buffer/MeOH=80:20 (v/v), and the flow rate was 0.5 mL/min. The system was calibrated with HA standards supplied by Dr. O. Wik (Pharmacia). HPMA copolymer conjugates were characterized by GPC on a Pharmacia FPLC with Superose analytical column, pH 7.4 PBS buffer was used as eluent with a flow rate of 0.4 ml/min. Cell viability in cell culture was determined by thiazoyl blue (MTT) dye uptake protocols measured at 540 nm, which was recorded on a BIO-RAD M-450 microplate reader (Hercules, Calif.). Laser scanning confocal microscopy was carried out on a Keller type Bio-Rad MRC 1024 with LASERSHARP acquisition software. Fluorescence images were taken using FITC settings with the 488 nm excitation line and a 522 nm 32 bandpass filter was used to collect the images.

### b) Preparation of Low Molecular Weight (LMW) HA and HA hydrazide derivative (HA-ADH)

LMW HA was obtained by the degradation of high molecular weight HA (1.5 MDa) in pH 6.5 phosphate-buffered saline (PBS) buffer (4 mg/mL) with HAse (10 U/mg HA) as previously described, and purified by dialysis against H₂ O³⁰. Hydrazide-derivatized HA (HA-ADH) was prepared^{30,51} using a modified purification method that gives preparations free of small molecules³⁰. In a representative example, LMW HA (50 mg) was dissolved in water to give a concentration of 4 mg/mL, and then a fivefold excess of ADH was added into the solution. The pH of the reaction mixture was adjusted to 4.75 by addition of 0.1 N HCl. Next, 1 equiv of EDCI was added in solid form. The pH of the reaction mixture was maintained at 4.75 by addition of 0.1 N HCl. The reaction was quenched by addition of 0.1 N NaOH to adjust the pH of reaction mixture to 7.0 for different reaction time. The reaction mixture was then transferred to pretreated dialysis tubing (Mw cutoff 3,500) and dialyzed exhaustively against 100 mM NaCl, then 25% EtOH/H₂O, and finally H₂O. The purity of HA-ADH was monitored by GPC. The purified polymer solution was then filtered through 0.2 μm cellulose acetate membrane, flash frozen, and lyophilized. The loading of ADH on the polymer backbone was determined by ¹H NMR in D₂O⁵¹. 37 mg of HA-ADH was obtained with 9 mol % and 18 mol % loading based on available carboxylates modified respectively, with the reaction time to be 12 min and 20 min.

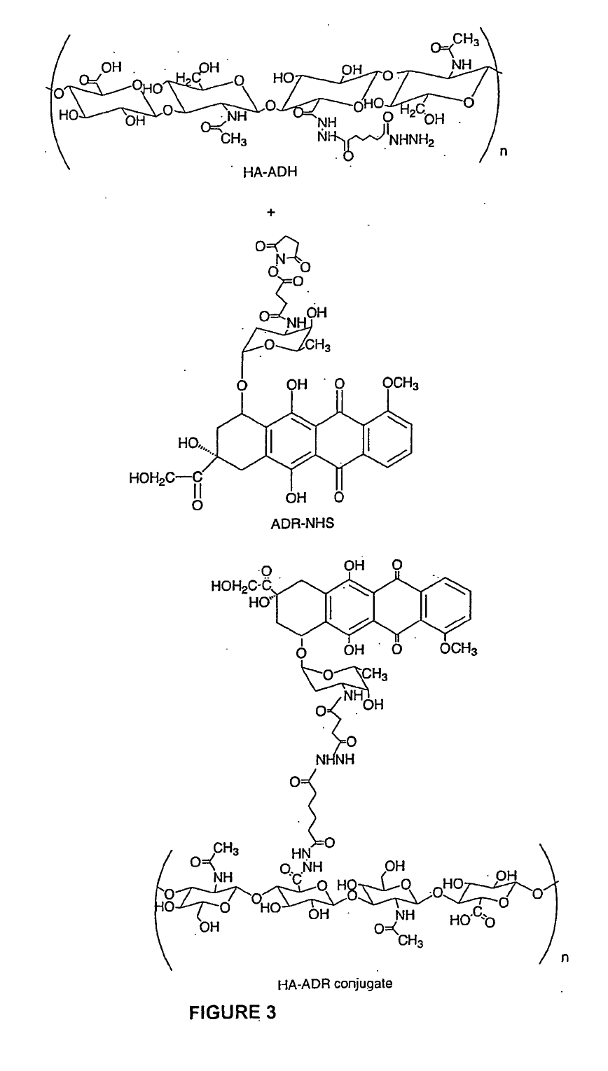

### c) Preparation of HA-DOX Conjugates (FIG. 3)

First, DOX was derived to be an active ester form (DOX-NHS). Briefly⁵², DOX at a 20-mg quality (34 μmol) was dissolved in 1.2 ml of anhydrous DMF, followed by 15 μl triethylamine and 3.8 mg succinic anhydride. The reaction was stirring at room temperature in dark for 24 hrs. DOX-hemisuccinate was purified by C₁₈ cartridge (Varian, Harbor City, Calif.) with methanol as the eluent.

Next, N-hydroxysuccinimido diphenyl phosphate (SDPP) was prepared from 10 mmol of diphenylphosphoryl chloride, 10 mmol of N-hydroxysuccinimide, and 10 mmol triethylamine in 6 mL of CH₂Cl₂ as previously described^{30,53}. Crude SDPP was titrated with ether, dissolved in ethyl acetate, washed (2×10 mL H₂O), dried (MgSO₄), and concentrated in vacuo to give SDPP with mp 89-90° C. (85%). To the solution of DOX-hemisuccinate and 18.5 mg (1.5 equiv) of SDPP in 2 ml DMF, was added with 60 μL (10 equiv) triethylamine. The reaction was stirred for 6 h at room temperature, and then concentrated in vacuo. The DOX-NHS ester was purified on a LH-20 column with methanol as the eluent.

HA-DOX conjugates were prepared by the conjugation of LMW HA-ADH and DOX-NHS. 50 mg HA-ADH (9 mol % and 18 mol %) was dissolved in 7 ml 3 mM pH 6.0 phosphate buffer, 2 mg DOX-NHS in 15 ml DMF was added to this solution under ice-water bath. The reaction was stirring at room temperature for 3 days. The HA-DOX conjugates were purified on a Sephadex G-25 column using PBS buffer as the eluent, following by dialysis against H₂O to remove the buffer salt. The DOX loading was determined by the absorption of UV spectrum at λ=484 nm.

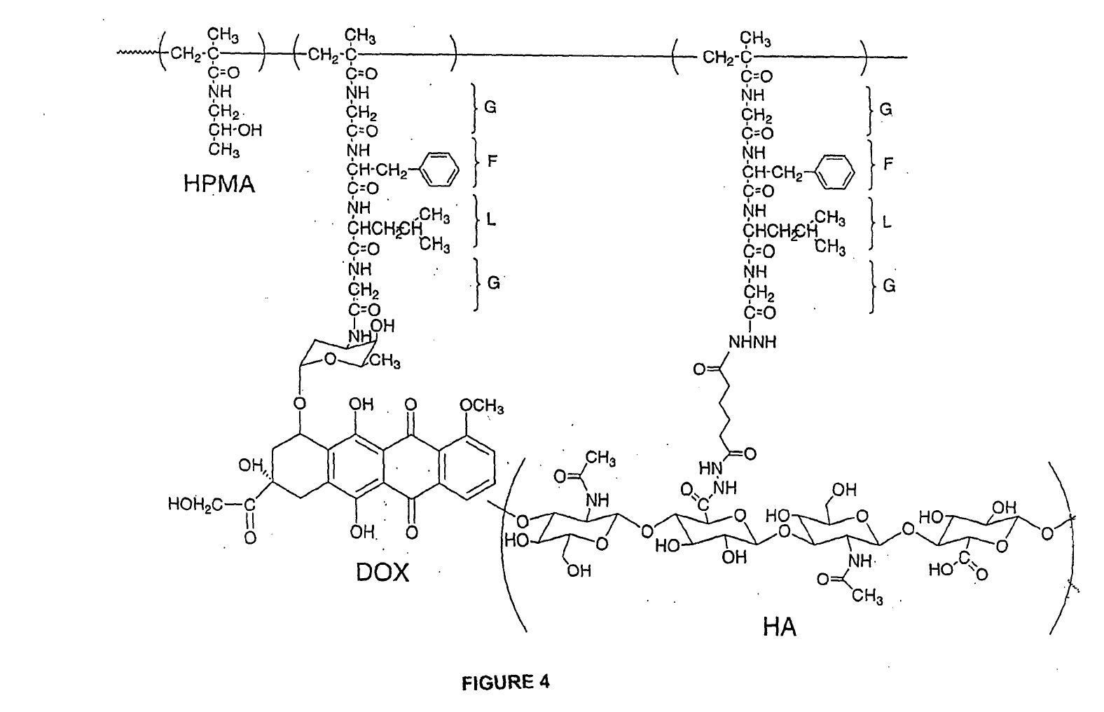

### d) Preparation of HPMA-HA-DOX Conjugates (FIG. 4)

The HPMA copolymer-bound DOX (HPMA-DOX or P(GFLG)-DOX; P is the HPMA copolymer backbone) was synthesized as previously described^{54,55}. A lysosomally degradable glycylphenylalanylleucylglycine (GFLG) spacer was used as the oligopeptide side chain. The conjugate was synthesized using a two step procedure⁵⁶. In the first step, the polymer precursor HPMA-(GFLG)-ONp was prepared by radical precipitation copolymerization of HPMA and N-methacryloylglycylphenylalanylleucylglycine p-nitrophenyl ester⁵⁵. The polymer precursor contained 7.1 mol % active ester groups (Mw=17,800, Mn=14,500). DOX was bound to the polymer precursor by aminolysis⁵⁷. 200 mg HPMA-(GFLG)-ONp and 21.9 mg doxorubicin (DOX) hydrochloride were dissolved in 1.0 ml DMSO, and 50 μl of Et3N was added. The mixture was stirred at room temperature for 1 hr, and precipitated in acetone/ether (3/1) mixture solvent. The red polymer solid was collected and washed with acetone, ether, dried under vacuum to give 210 mg product. The HPMA-(GFLG)-DOX-ONp conjugate contained 1.1 mol % of DOX.

HPMA-HA-DOX conjugates were prepared by the conjugation of HA-ADH (9 mol % and 18 mol % hydrazide modification) to the above HPMA-(GFLG)-DOX-ONp with ONp residue. For example, 90 mg HPMA-(GFLG)-DOX-ONp copolymer-drug conjugate prepared previously was dissolved in 2.0 ml DMSO, and 90 mg HA-ADH of 18 mol % hydrazide modification was dissolved in 1.0 ml water and 2.0 ml DMSO. The two solutions were mixed together and stirred it overnight at room temperature. Aminoethanol (100 μl) was added to destroy unreacted ONp active ester. The HPMA-HA-ADR conjugate was isolated and purified by gel filtration on a Sephadex LH-20 column twice with methanol as eluent. The solvent was removed under vacuum, and the residue was dissolved in distilled water and lyophilized. The DOX loading was determined by the absorption of UV spectrum at λ=484 nm. HA composition was calculated by mass balance.

### e) In Vitro Cell Culture

The cytotoxicity of HA-DOX and HPMA-HA-DOX conjugates against HBL-100, SKOV-3 and HCT-116 cells was determined using a 96-well plate format in quadruplicate with increasing doses range from 0.001-10 mg/mL of DOX equivalent. Each well contained approximately 20,000 cells in 200 μL cell culture media. Thus, a 2-μL aliquot of the stock solution was added to each well, and cells were continuously incubated at 37° C., 5% CO₂ for 3 days with the test substance, and cell viability was determined using MTT dye uptake at 540 nm. Response was graded as percent live cells compared to untreated controls⁵⁸. Dose-response curves were constructed, an d the concentration necessary to inhibit the growth of the cells by 50% relative to the non-treated control cells (IC₅₀ dose) was determined.

Internalization of HPMA-HA-DOX conjugates by cancer cells by confocal fluorescence microscopy. SKOV-3 cells were incubated in a cell culture flask, harvested by trypsinization, and transferred into a 8-well cell culture slide. 20,000 cells were seeded in each well of the slide and cultured for 48 hr. The cultured medium was replaced with medium containing HPMA-HA-DOX conjugates, the concentration was adjusted to 50 μg/ml of HA equivalent. Meanwhile, HPMA-DOX conjugate with equal amount of DOX drug to HPMA-HA-DOX was used as a control. Cells were cultured with the conjugates for various time intervals. Unbound conjugate was removed by washing the cell layer 3 times with DPBS. Cells were fixed with 3% paraformaldehyde for 10 min at room temperature and washed again with DPBS. Internalized HPMA-HA-DOX conjugate was visualized by fluorescence images taken with the confocal microscopy.

In the cell surface binding experiment, cells were incubated with the HPMA-HA-DOX conjugate at 0° C. for 2hr (a condition under which no internationalization occurs), followed by the DPBS washing and paraformaldehyde fixing described above. The cell surface binding conjugate was determined by the fluorescence images.

Fluorescence microscopy. Cells were examined by using an inverted microscope (Nikon) and a Bio-Rad (Hercules, Calif.) MRC 1024 laser scanning confocal microscope. Cell images were collected by using a ×60 oil immersion objective, no postacquisition enhancement of images was performed. DOX fluorescene image acquisition was accumulated via the BHS block of filters (excitation 488 nm and emission through a 522 nm 32 bandpass filter). A coverslip was mounted on a microscope slide containing fixed cells with ProLong Antifade Kit (Molecular Probes, Eugene, Org.) as the mounting medium. Fluorescence images were scaled to 256 gray levels.

### f) Preparation of HA-DOX Conjugates

The hydrazide method to make the HA-ADH derivatives^{30,51,59} allows attachment of reporter molecules, drugs, crosslinkers, and combinations of these moieties to HA^{23,24}. LMW HA was generated in this study for three reasons: (i) proton NMR allowed rapid quantification of the modification, (ii) LMW HA and its derivatives give injectable, non-viscous solution at concentrations up to 10 mg/mL, and (iii) LMW HA has a longer plasma half-life and is readily cleared by renal ultrafiltration. The LMW HA was prepared by partial degradation of high molecular weight HA (1.5 MDa) with testicular HAse⁶⁰ in pH 6.5 PBS buffer at 37° C. The final size of LMW HA was characterized by GPC analysis: Mₙ=3,883, M_{w}=11,199, and molecular dispersity (DP)=2.88. Next, HA-ADH with different ADH loadings were prepared by carbodiimide coupling chemistry^{30,31}, in which the extent of ADH modification was controlled through use of specific molar ratios of hydrazide, carboxylate equivalents, and carbodiimide. The purity and molecular size distribution of the HA-ADH were measured by GPC, and the substitution degree of ADH was determined by the ratio of methylene hydrogens to acetyl methyl protons as measured by ¹H NMR⁵¹. HA-ADH with ADH loadings of 9 mol % and 18 mol % were obtained and used in preparing the HA-DOX and HPMA-HA-DOX conjugates.

Furthermore, HA-DOX conjugates was synthesized by the conjugation of HA-ADH to the activated DOX-NHS ester to give a non-cleavable hydrazide linkage between the DOX drug and the HA polymer carrier. The HA-DOX conjugates were purified by gel filtration on a Sephadex G-25 column using PBS buffer as the eluent, following by dialysis against H₂O. The DOX loading was determined by the UV spectrum at λ=484 nm. The DOX composition of the HA-DOX conjugates used in the in vitro cytotoxicity test were 2.3 wt % and 3.5 wt % which were made from 9 mol % and 18 mol % ADH loading of HA-ADH, respectively.

### g) Preparation of HPMA-HA-DOX Conjugates

This cell targeted delivery system was designed with HA on the side chain of the HPMA copolymer serving as a targeting moiety to cancer cell surface, and DOX linked to the polymer carrier through an lysosomal enzyme degradable peptide linkage¹². HPMA-HA-DOX conjugates were synthesized by the conjugation of HA-DOX with HPMA-DOX copolymer with active ONp residue. HA-ADH with 9 mol % and 18 mol % hydrazide modification were used in the conjugation. The conjugates were purified by gel filtration on a Sephadex LH-20 column. HA loading was determined by mass balance. The DOX loading was determined by the UV spectrum at λ=484 nm. HPMA-HA-DOX conjugates made from 18 mol % HA-ADH gave 36 wt % HA and 3.3 wt % DOX with molecular weight of Mw=35,000 and Mn=19,000. HPMA-HA-DOX conjugates made from 9 mol % HA-ADH gave 17 wt % HA and 3.2 wt % DOX with molecular weight of Mw=18,000 and Mn=14,000.

### h) Cytotoxicity Assay of HA-DOX and HPMA-HA-DOX Conjugates

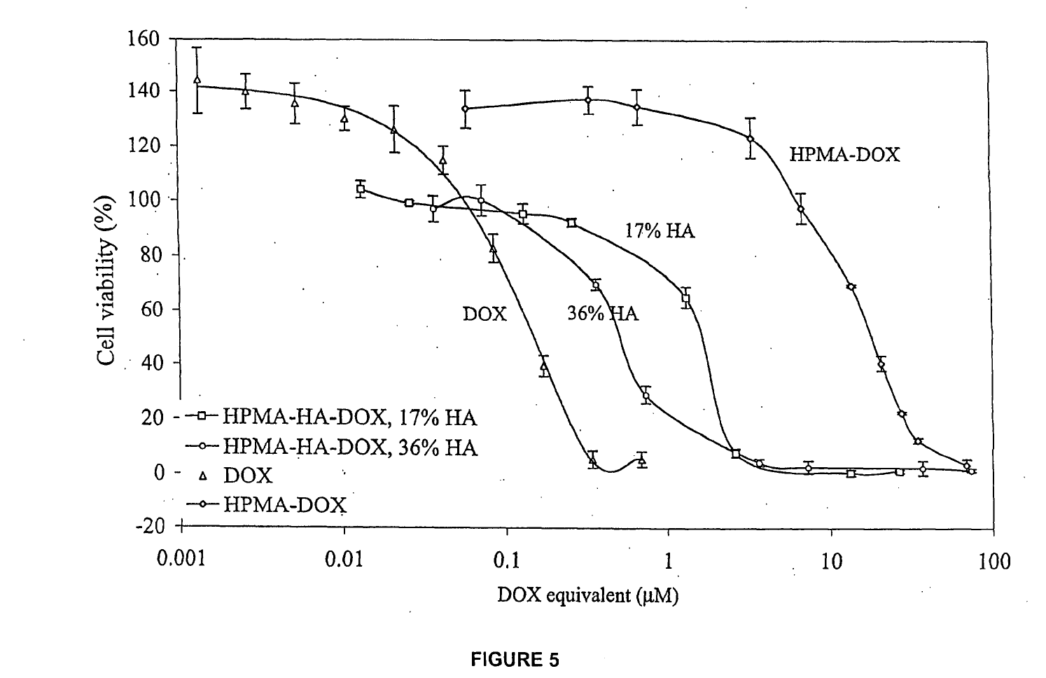

Free DOX drug and non-targeted HPMA-DOX and targeted HA-DOX, HPMA-HA-DOX conjugates were assessed for their dose-dependent growth inhibitory effect on human breast cancer HBL-100 cells, human ovarian cancer SKOV-3 cells and human colon cancer HCT-116 cells which have been reported to overexpress HA receptors on the tumor cell surface. Cells were exposed to various DOX concentration (DOX equivalent for polymer-drug conjugates) to determine the concentration necessary to inhibit the tumor cell growth by 50% relative to non-treated control cells (IC₅₀, dose). Typical curves describing the dependence of cell viability on the concentration of DOX equivalent covalently bound to the polymer conjugates, were presented in FIG. 5. The IC₅₀ doses for the free DOX drug and the conjugates were listed in Table 1. From these results it is clear that DOX attached to a non-targeted polymer carrier (HPMA-DOX) markedly decrease the cytotoxicity of DOX drug. For SKOV-3 cells, the IC₅₀ doses increase from 0.92 μM for free DOX drug to 58.2 μM for HPMA-DOX. These increases probably reflect the different mechanisms of cell uptake (free diffusion for free DOX drug vs. endocytosis for DOX-polymer conjugates) resulting in different intracellular drug concentration. Targeted HPMA-HA-DOX conjugates which enter cells by receptor-mediated endocytosis, nearly restored the original low IC₅₀ dose for DOX drug. The IC₅₀ doses against HBL-100 cells were 0.52 μM and 1.67 μM for the targeted HPMA-HA-DOX conjugates with 36 wt % and 17 wt % HA loading, respectively, in comparison of the 18.7 μM for the non-targeted HPMA-DOX conjugate and 0.15 μM for free DOX drug. Against each cell line overexpressed HA receptors on cell surface, the cytotoxicity of targeted HPMA-HA-DOX conjugates had a magnitude increase over the non-targeted HPMA-DOX conjugate.

However, for the HA-DOX conjugate system, the cytotoxicity of the conjugates were even slightly higher than the non-targeted HPMA-DOX conjugate. The IC₅₀ doses against SKOV-3 cells were 157 μM and 141 μM for HA-DOX conjugates, comparing to 58.2 μM for non-targeted HPMA-DOX conjugate, and 9.2 μM for targeted HPMA-HA-DOX conjugate (36 wt %). Two possible factors would contribute to the loss of cytotoxicity: the conjugation decreases the activity of DOX drug; the non-cleavable hydrazide linkage between DOX and HA polymer carrier. From our previous study, the cytotoxicity HA-Taxol conjugates with esterase cleavable linkage between Taxol drug and HA polymer carrier had a comparable value to free Taxol drug in cell culture against SKOV-3 cells³⁰.

TABLE 1
Cytotoxicity of free DOX drug, HA-DOX conjugates
and HPMA-HA-DOX conjugates against
SKOV-3 cells in vitro.

| | IC₅₀ (μM) of DOX equivalent | | |
|---|---|---|---|
| | HBL-100 | SK-OV-3 | HCT |
| Drugs | cells | cells | 116 cells |
| DOX | 0.15 | 0.92 | 0.35 |
| HA-DOX (2.3 wt % DOX) | 100 | 157 | 140 |
| HA-DOX (3.5 wt % DOX) | 75.5 | 141 | 62.0 |
| HPMA-DOX | 18.7 | 58.2 | 56.6 |
| HPMA-HA-DOX (36 wt % HA) | 0.52 | 9.2 | 4.32 |
| HPMA-HA-DOX (17 wt % HA) | 1.67 | 10.3 | 5.66 |

### i) Cell Binding and Uptake of HPMA-HA-DOX Conjugates

Several different fluorescently-labeled HA derivatives have been prepared in order to study receptor-mediated cellular uptake. Previously, fluorescein-HA was employed to study HA uptake in a variety of systems, e.g., cells expressing CD44 variants^{40,41,61-64}, uptake by tumor cells for correlation with metastatic potential^{50,65}, internalization by chondrocytes⁴⁶, and as a measure of liver endothelial cell function⁶⁶. Most recently, RHAMM-mediated uptake and trafficking of HA by transformed fibroblasts⁶⁷ was observed with Texas Red-HA, and BODIPY-labeled HA was employed to distinguish HA uptake in cancer vs. untransformed cell-lines^{30,31} .

In order to correlate the receptor-mediated endocytosis of conjugates by cells with their cytotoxicity, the cell binding and uptake of the targeted HPMA-HA-DOX conjugates were followed by the fluorescene microscopy using the intrinsic fluorescence of DOX. Cells were cultured in the presence of HPMA-HA-DOX conjugates of 50 μg/ml HA equivalent for various period of time, afterwards the amount of material internalized and bound to cell surface was visualized by confocal fluorescene microscopy.

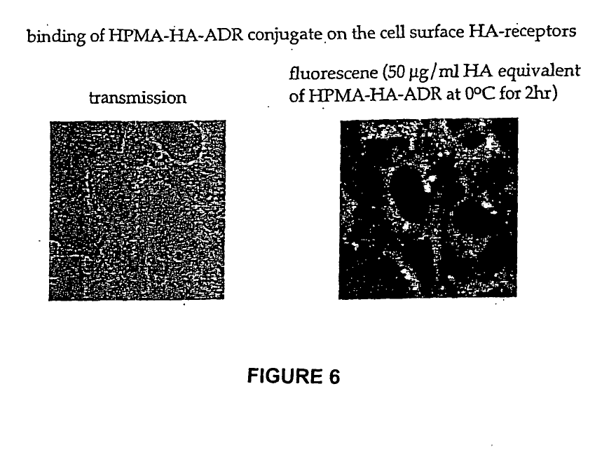

SKOV-3 Cells chilled to 0° C. was incubated with HPMA-HA-DOX for 2hr. After fixing and washing, a well-developed cluster of cells was chosen for the fluorescence microscope analysis. Cells were sectioned optically using confocal microscopy, fluorescence images were taken via the BHS block of filters of excitation 488 nm and emission 522 nm, along with the transmission images. FIG. 6 provided a particularly dramatic illustration of the initial binding of the HPMA-HA-DOX conjugate on the SKOV-3 cells surface where the overexpressed HA binding receptor-CD44 located. The anchoring of the targeted HPMA-HA-DOX on the cell surface prior to the cellular uptake through the specific binding between HA and HA binding proteins, provides the opportunity of the enhanced internalization of the polymer conjugates by receptor-mediated endocytosis.

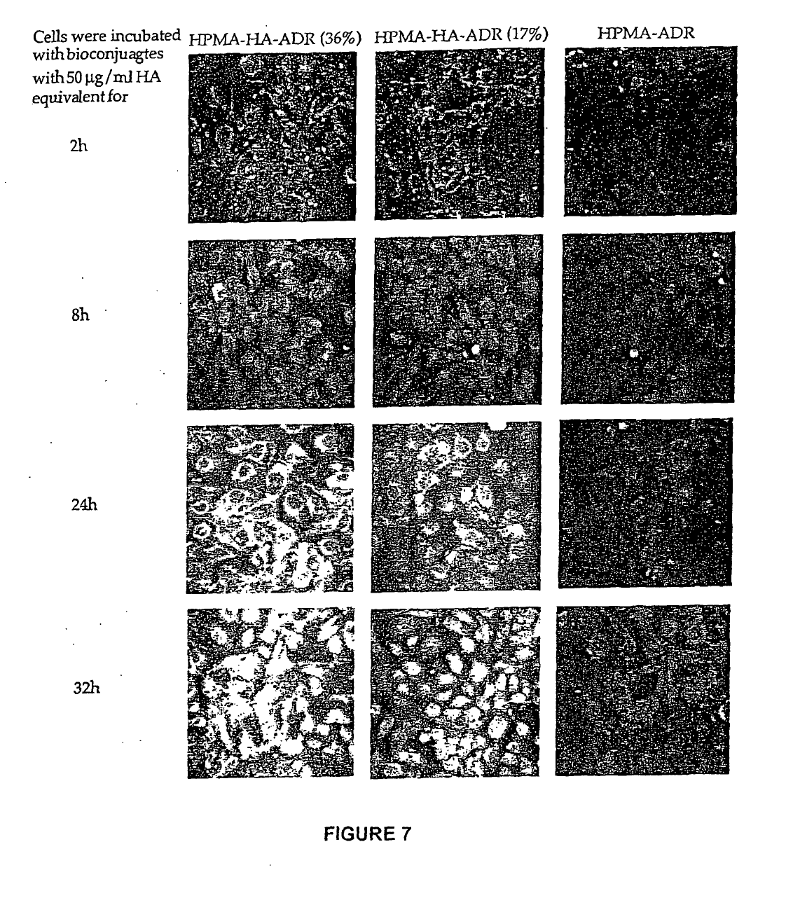

In addition, the internalization of polymer conjugates directly determined the cytotoxicity of conjugate system. Thus, with the intrinsic fluorescence of DOX, the cellular uptake of the targeted HPMA-HA-DOX conjugates were also followed by the confocal fluorescence microscopy. SKOV-3 cells were incubated with the HPMA-HA-DOX conjugates (36 wt % and 17 wt % HA loading) of 50 μg/ml HA equivalent for various intervals, before the fluorescence images were taken. The non-targeted HPMA-DOX of equal amount of DOX equivalent was used as a control. Confocal fluorescence images of HPMA-HA-DOX uptake by SKOV-3 cells were presented in FIG. 7. Initially the 2 hr images, HPMA-HA-DOX polymer conjugates could be seen mainly on the cell membrane; over the course of 8 hr, it was gradually taken up into the cells. 24 hr and 32 hr later, cells showed the polymer conjugates in most subcellular compartments. The uptake of HPMA-HA-DOX conjugate with 36 wt % HA loading was rapid than the conjugate with 17 wt % HA loading, however, no significant difference was observed. In the control of non-targeted cellular uptake of HPMA-DOX, the fluorescence inside cells was gradually increase along with the incubation time of cells with the polymer conjugate. However, very weak fluorescence (polymer conjugate) was observed even after 32 hr incubation, in comparison of the targeted HPMA-HA-DOX system. The uptake of HPMA-HA-DOX into HBL-100 cells and HCT-116 cells occurred with a similar appearance and time course. These images provided a particularly dramatic illustration of the initial binding of the targeted HPMA-HA-DOX conjugates onto the tumor cell surface, following by rapid endocytosis via HA receptor-mediated pathways. HA incorporated into HPMA-DOX conjugates significantly increase the efficiency of the endocytosis process by cancer cells. The trafficking of cellular binding and uptake of HPMA-HA-DOX conjugates by confocal fluorescence images is consistent with the cytotoxicity results, and provides the further support for the increase cytotoxicity of targeted HPMA-HA-DOX conjugates of which the enhanced internalization of polymer conjugates mediated through an HA-specific, receptor-mediated process comparing to the non-targeted HPMA-DOX system.

In summary, the data reported herein indicate that the cytotoxicity of HPMA-HA-DOX polymer conjugates requires cellular uptake of the bioconjugate followed by the release of the active free DOX drug by the lysosomal enzyme cleavage of the GFLG tetra-peptide spacer. Targeting of a variety of anti-cancer agents to tumor cells and tumor metastases could be achieved by receptor-mediated uptake of an HA containing-anti-cancer agent conjugate, followed by the intracellular release of the active drug and subsequent cell death. The ability to “seek and destroy” micrometastases is one of the most compelling and attractive potential outcomes for the disclosed HA containing-anti-tumor bioconjugates.

### j) In Vitro Cytoxicity of HPMA-HA-DOX Conjugates

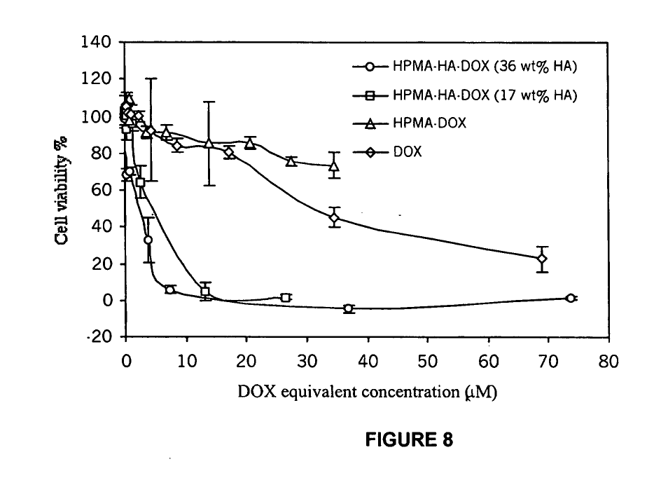

The in vitro cytotoxicity of HPMA-HA-DOX with 17% and 36% HA loading against cultured prostate cancer cell line DU-145 was examined. FIG. 8 depicts the in vitro cytotoxicity results of the HPMA-HA-DOX bioconjugates. The cytotoxicity of targeted HPMA-HA-DOX bioconjugates were dramatically higher than non-targeted HPMA-DOX conjugate (Table 2), and 8- to 12-fold higher than the free DOX drug against this prostate cancer cell-line. These data indicate that HPMA-HA-DOX bioconjugate can be used as a specific prostate cancer macromolecular chemotherapeutic agent.

TABLE 2
In vitro Cytotoxicity of free DOX drug, HA-DOX conjugates
and HPMA-HA-DOX conjugates against human
prostate cancer cell-line DU-145.

| | IC₅₀ (μM) of DOX |
|---|---|
| | equivalent against DU-145 |
| DOX | 31.5 |
| HPMA-DOX | >100.0 |
| HPMA-HA-DOX (36 wt % HA) | 2.4 |
| HPMA-HA-DOX (17 wt % HA) | 4.7 |

1. A compound comprising an anti-cancer agent, a carrier molecule, and hyaluronic acid or a derivative thereof, wherein the anti-cancer agent, the carrier molecule, and the hyaluronic acid or a derivative thereof are attached to one another via a covalent bond.

2. The compound of claim 1, wherein the anti-cancer agent comprises a cytotoxic agent, a chemotherapeutic agent, a cytokine, an antitubulin agent, a radioactive isotope, a combretastatin antagonists, a calcium ionophore, a calcium-flux inducing agent, or a combination thereof.

3. The compound of claim 1, wherein the anti-cancer agent comprises 5-fluorouracil, 9-aminocamptothecin, amine-modified geldanomycin, Taxol®, vincristine, vinblastine, vinorelbinie, and vindesine, calicheamicin, QFA, BCNU, streptozoicin, neomycin, podophyllotoxin, TNF-alpha, .alphaᵥbeta₃ colchicinie, or a combination thereof.

4. The compound of claim 1, wherein the anti-cancer agent is doxorubicin.

5. The compound of claim 1, wherein the carrier molecule comprises a macromolecule of at least 5,000 daltons.

6. The compound of claim 1, wherein the carrier molecule comprises a macromolecule having a molecular weight of from 10,000 daltons to 25,000 dalton.

7. The compound of claim 1, wherein the carrier molecule comprises a polymer produced by the polymerization of an ethylenically unsaturated monomer.

8. The compound of claim 7 wherein the monomer is an acrylate or methacrylate.

9. The compound of claim 7 wherein the monomer is N-(2-hydroxypropyl)methacrylamide.

10. The compound of claim 1, wherein the derivative of hyaluronic acid comprises hyaluronic acid modified with a dihydrazide compound.

11. The compound of claim 10 wherein the dihydrazide compound is adipic dihydrazide.

12. The compound of claim 1, wherein the anti-cancer agent is directly attached to the carrier molecule by a covalent bond.

13. The compound of claim 1, wherein the anti-cancer agent is indirectly attached to the carrier molecule by a linker, wherein the anti-cancer agent and the carrier molecule are individually attached to the linker via a covalent bond.

14. The compound of claim 13 wherein the linker comprises a peptide.

15. The compound of claim 1, wherein the carrier molecule is directly attached to the hyaluronic acid or the derivative thereof by a covalent bond.

16. The compound of claim 1, wherein the carrier molecule is attached to the hyaluronlic acid or the derivative thereof by a covalent bond, and the hyaluronic acid or the derivative thereof is attached to the anti-cancer agent by a covalent bond.

17. The compound of claim 1, wherein the carrier molecule is attached to the anti-cancer agent by a covalent bond, and the anticancer agent is attached to the hyaluronic acid or the derivative thereof by a covalent bond.

18. The compound of claim 1, wherein the anti-cancer agent is doxorubicin, the carrier molecule is a polymer of N-(2-hydroxypropyl)methacrylamide, and the hyaluronic acid modified with adipic dihydrazide.

19. A pharmaceutical composition comprising the compound of claim l and a pharmaceutically acceptable carrier.

20. A composition comprising two or more compounds of claim 1.

21. A process for making a compound, comprising reacting an anti-cancer agent, a carrier molecule, and hyaluronic acid or the derivative thereof with each other to produce the compound.

22. The process of claim 21 comprising (1) reacting the anti-cancer agent with the carrier molecule to produce a carrier/anti-cancer molecule and (2) reacting the carrier/anti-cancer molecule with hyaluronic acid or the derivative thereof.

23. The process of claim 22 wherein the carrier molecule comprises a linker comprising a group that can react with the anti-cancer agent.

24. The process of claim 23 wherein the derivative of hyaluronic acid comprises the reaction product between hyaluronic acid and adipic dihydrazide.

25. The process of claim 24 wherein the anticancer agent is doxorubicin and the carrier molecule is a polymer of N-(2-hydroxypropyl)methacrylamide.

26. The process of claim 21 comprising (1) reacting the anti-cancer agent with hyaluronic acid or the derivative thereof to produce an anti-cancer/hyaluronic acid molecule; (2) reacting the anti-cancer agent with the carrier molecule to produce a carrier/anti-cancer molecule; and (3) reacting the anti-cancer/hyaluronic acid molecule with the carrier molecule/anti-cancer molecule.

27. The process of claim 26 wherein the derivative of hyaluronic acid comprises the reaction product between hyaluronic acid and adipic dihydrazide.

28. The process of claim 27 wherein the anticancer agent is doxorubicin and the carrier molecule is a polymer of N-(2-hydroxypropyl)methacrylamide.

29. The compound produced by the process of claim 21.

30. The compound produced by the process of claim 22.

31. The compound produced by the process of claim 25.

32. The compound produced by the process of claim 26.

33. The compound produced by the process of claim 28.

34. The compound of claim 1, wherein the compound comprising the anti-cancer agent, a carrier molecule, and hyaluronic acid or a derivative thereof is taken up by a cell with a greater efficiency than a compound comprising the anti-cancer agent and the carrier molecule alone.

35. The compound of claim 1, wherein the compound comprising the anti-cancer agent, a carrier molecule, and hyaluronic acid or a derivative thereof is taken up by a cell with greater cellular specificity than a compound comprising the anti-cancer agent and the carrier molecule alone.

36. A method of inhibiting cancer cell proliferation comprising administering the compound of claim 1 to a cancer cell.

37. A method of treating a patient with cancer comprising administering the compound of claim 1 to the patient.

38. A method of treating a patient comprising administering the compound of claim 1 to the patient.
